(19) United States
(12) Patent Application Publication
(10) Pub. No.: US 2023/0129075 A1
(43) Pub. Date: Apr. 27, 2023

(54) ERROR SUPPRESSION IN GENETIC SEQUENCING

(71) Applicant: St. Jude Children's Research Hospital, Memphis, TN (US)

(72) Inventors: Xiaotu Ma, Memphis, TN (US); Eric M. Davis, Memphis, TN (US)

(21) Appl. No.: 17/792,284

(22) PCT Filed: Jan. 13, 2021
(86) PCT No.: PCT/US2021/013267

§ 371 Date: Jul. 12, 2022

Related U.S. Application Data
(60) Provisional application No. 62/960,476, filed on Jan. 13, 2020.

(51) Int. Cl. G16B 30/00; C12Q 1/6869

(52) U.S. Cl. CPC G16B 30/00; C12Q 1/6869

(57) ABSTRACT

A method for measuring and suppressing errors within instrument (sequencer) of targeted next generation sequencing workflow are described herein.

### CROSS-REFERENCE TO RELATED APPLICATION

This application claims the benefit of U.S. Provisional Application No. 62/960,476, filed on Jan. 13, 2020, the entirety of which is hereby incorporated by reference.

### BACKGROUND

Next generation sequencing (NGS) plays an ever-increasingly important role in biomedicine in elucidating the genetic makeups of cell populations. Sequencing errors are key confounding factors for detecting low-frequency genetic variants that are important for cancer molecular diagnosis, treatment, and surveillance using deep NGS. However, there is a lack of comprehensive understanding of errors introduced at various steps of a conventional NGS workflow, such as sample handling, library preparation, PCR enrichment, and sequencing. For example, an error rate may be the product of both PCR errors and instrument (i.e., sequencer) errors, and it is currently unknown how to separate these error sources. Deep DNA sequencing NGS technology holds great promise but the sequencing accuracy remains a bottleneck for these applications However, currently there is no method to measure errors induced in sequencers. As a result, it remains challenging to make informed decisions on platform options for deep sequencing applications, to diagnose problems in instrumentation, and to further improve the instrumentation. As a result, it remains an open question on how to measure errors introduced in the sequencer. The methods and systems described herein address this need.

### SUMMARY

It is to be understood that both the following general description and the following detailed description is merely an example and is explanatory only and is not restrictive. Described herein is a decision support system and method to determine errors in genetic sequencing associated with a device (e.g., the sequencer). The decision support system and method applies statistical methods and graphic visualizations to features extracted from sequencing output and the characteristics associated with samples, to provide practitioners estimates on errors introduced within one or more sequencers of the targeted next generation sequencing workflow. As a result, various experimental parameters such as instrument calibration, flowcell quality, tile quality can be readily assessed with high precision. Such parameters can also be used to re-calibrate different components of the sequencing instrument as mentioned above to improve its performance. The method can also detect instrument-level and consumable-level aberrations and perform corrections on such aberrations for existing data to achieve optimized results. In turn, this method plays important role for achieving much-enhanced accuracy for sensitive detection of low frequency variants. In addition, the techniques described herein can suppress sequencer errors in addition to previously discovered error sources. A method is described for measuring and suppressing errors in next-generation genetic sequencing (NGS). The method may comprise associating types of sequencing errors with devices (e.g., sequencers) involved in the sequencing process, identifying machines that produce particular errors, error rates, or types of errors associated with particular sequences, and removing problematic sequences and/or error types. The methods may comprise determining patterns of sequencer errors including: 1) the overall sequencer error rate; 2) at the flow-cell level, error rates are elevated in the bottom surface; 3) almost all flow cells have a small fraction of random tiles with a dramatically elevated error rate; 4) the elevated error rates appear to be enriched in some reaction cycles; 5) removal of certain reaction cycles yields lower error rates at some genomic loci, so that A>C, A>T, and C>G error types have reduced error rates; and 6) sequencer error have a pattern markedly distinct from PCR errors. The methods incorporate a general-purpose algorithm, termed SequencErr, to computationally determine and suppress sequencer errors. This summary is not intended to identify critical or essential features of the disclosure, but merely to summarize certain features and variations thereof. Other details and features will be described in the sections that follow.

### BRIEF DESCRIPTION OF THE DRAWINGS

FIG. 1 shows an example system;

FIG. 2 shows an example process flow;

FIG. 3 shows an example process flow;

FIG. 4 shows an example error profile;

FIG. 5A shows an example error profile;

FIG. 5B shows an example error profile;

FIG. 5C shows an example error profile;

FIG. 6 shows an example error profile;

FIG. 7 shows an example error profile;

FIG. 8 shows an example flow cell process flow;

FIG. 9 shows an example sequencing process;

FIG. 10 shows an example process flow;

FIG. 11 shows an example process flow;

FIG. 12 shows an example process flow;

FIG. 13 shows an example process flow;

FIG. 14 shows an example process flow;

FIG. 15 shows an example process flow;

FIG. 16 shows an example method; and

FIG. 17 shows an example computing environment for implement the disclosed methods and systems.

### DETAILED DESCRIPTION

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another configuration includes from the one particular value and/or to the other particular value. When values are expressed as approximations, by use of the antecedent “about,” it will be understood that the particular value forms another configuration. It will be further understood that the endpoints of each of the ranges are significant both in relation to the other endpoint, and independently of the other endpoint.

“Optional” or “optionally” means that the subsequently described event or circumstance may or may not occur, and that the description includes cases where said event or circumstance occurs and cases where it does not.

Throughout the description and claims of this specification, the word “comprise” and variations of the word, such as “comprising” and “comprises,” means “including but not limited to,” and is not intended to exclude other components, integers or steps. “Exemplary” means “an example of” and is not intended to convey an indication of a preferred or ideal configuration. “Such as” is not used in a restrictive sense, but for explanatory purposes.

It is to be understood that when combinations, subsets, interactions, groups, etc. of components are described that, while specific reference of each various individual and collective combinations and permutations of these may not be explicitly described, each is specifically contemplated and described herein. This applies to all parts of this application including, but not limited to, steps in described methods. Thus, if there are a variety of additional steps that may be performed it is understood that each of these additional steps may be performed with any specific configuration or combination of configurations of the described methods.

As will be appreciated by one skilled in the art, hardware, software, or a combination of software and hardware may be implemented. Furthermore, a computer program product on a computer-readable storage medium (e.g., non-transitory) having processor-executable instructions (e.g., computer software) embodied in the storage medium. Any suitable computer-readable storage medium may be utilized including hard disks, CD-ROMs, optical storage devices, magnetic storage devices, memresistors, Non-Volatile Random Access Memory (NVRAM), flash memory, or a combination thereof.

Throughout this application reference is made to block diagrams and flowcharts. It will be understood that each block of the block diagrams and flowcharts, and combinations of blocks in the block diagrams and flowcharts, respectively, may be implemented by processor-executable instructions. These processor-executable instructions may be loaded onto a general purpose computer, special purpose computer, or other programmable data processing apparatus to produce a machine, such that the processor-executable instructions which execute on the computer or other programmable data processing apparatus create a device for implementing the functions specified in the flowchart block or blocks.

These processor-executable instructions may also be stored in a computer-readable memory that may direct a computer or other programmable data processing apparatus to function in a particular manner, such that the processor-executable instructions stored in the computer-readable memory produce an article of manufacture including processor-executable instructions for implementing the function specified in the flowchart block or blocks. The processor-executable instructions may also be loaded onto a computer or other programmable data processing apparatus to cause a series of operational steps to be performed on the computer or other programmable apparatus to produce a computer-implemented process such that the processor-executable instructions that execute on the computer or other programmable apparatus provide steps for implementing the functions specified in the flowchart block or blocks.

Accordingly, blocks of the block diagrams and flowcharts support combinations of devices for performing the specified functions, combinations of steps for performing the specified functions and program instruction means for performing the specified functions. It will also be understood that each block of the block diagrams and flowcharts, and combinations of blocks in the block diagrams and flowcharts, may be implemented by special purpose hardware-based computer systems that perform the specified functions or steps, or combinations of special purpose hardware and computer instructions.

This detailed description may refer to a given entity performing some action. It should be understood that this language may in some cases mean that a system (e.g., a computer) owned and/or controlled by the given entity is actually performing the action.

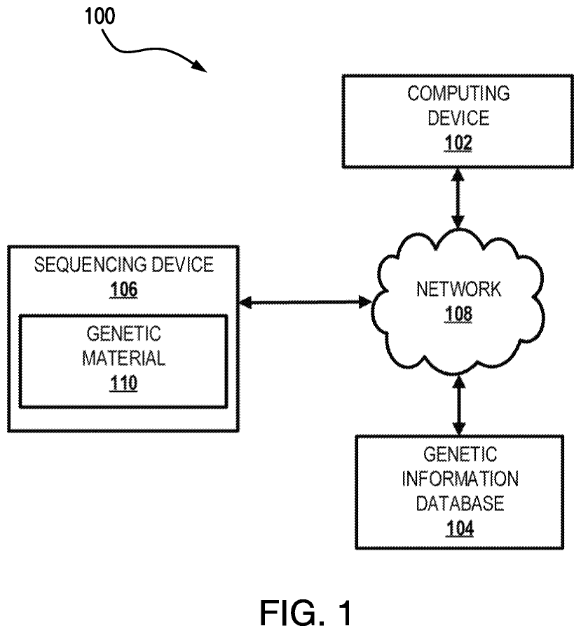

FIG. 1 shows an example system 100 which may facilitate execution of the present methods. The system 100 may comprise a computing device 102. The computing device 102 may be configured to send and receive data (e.g., genetic information). For example, the computing device 102 may be a computing device such as a computer, server, laptop, smart phone, camera, or any other device capable of receiving, storing, generating, processing, or sending data (e.g., the genetic information). The computing device 102 may be configured to send and receive data to/from the sequencing device and/or a genetic information database (GID) 104 and/or a sequencing device 106 via a network 108.

The genetic information database 104 may comprise a computing device. For example, the GID 104 may be a computing device such as a computer, server, laptop, smart phone, camera, or any other device capable of receiving, storing, generating, processing, or sending data (e.g., the genetic information). The GID 104 may be configured to send/receive data from the computing device 102 and/or the sequencing device 106 via the network 108. The genetic information database 104 may store the genetic information. The genetic information may comprise DNA. The genetic information may comprise a DNA sequence. The genetic information may comprise a dataset. The genetic information may comprise a library. The genetic information may comprise a sample. The genetic information may comprise a cell line. For example, DNA may be extracted from stored samples by using either a QIAamp DNA Blood Mini Kit (QIAGEN cat #51106) or a DNeasy Blood & Tissue Kit (cat #69506). For example, one or more deep sequencing datasets may be generated by a research hospital, university, private company, or any other organization. For example, the one or more datasets may be associated with St. Jude Children's Research Hospital (St. Jude), HudsonAlpha Institute of Biotechnology (HAIB), and WuXiNextCode. For example, whole-exome sequencing datasets may be received from Broad Institute (BI) and/or Baylor College of Medicine (BCM). For example datasets may be acquired by querying the genetic information database 104. For example, relevant datasets from a sequence read archive (“SRA) may be determined. For example, the National Center for Biotechnology Information Sequence Reach Archive (e.g., the NCBI SRA) may be queried using “(NovaSeq) AND “Homo sapiens” [orgn:_txid9606]” and “(NextSeq) AND “Homo sapiens” [orgn:_txid9606]” and results may be downloaded with requirement that the datasets are paired-end DNA sequencing. The datasets may be manually inspected as well.

The genetic information may comprise one or more datasets. For example, the one or more datasets may comprise an ultra-deep sequencing (1,000,000× depth) dataset. The one or more datasets may, for example, be publicly available datasets such as the COLO829 dilution datasets published previously, where exactly the same DNA libraries were sequenced on two different NovaSeq instruments, (in Computational Biology Genomics Laboratory, St. Jude Children's Research Hospital, Memphis, Tenn.) and (in HudsonAlpha Institute of Biotechnology, Huntsville, Ala.). The datasets may be generated by one or more sequencers. For example, the one or more sequencers may comprise a HiSeq sequencer, a NovaSeq sequencer, and/or a NextSeq sequencer. The one or more datasets may comprise a benchmark dataset (e.g., a truth dataset).

In an embodiment, the sequencing device 106 may determine the genetic information. The genetic information may comprise a sequence. For example, the genetic information may be determined based on a sample input. Exemplary platforms include HiSeq, NovaSeq, and NextSeq, which are Illumina platforms. The aforementioned are merely exemplary and one of skill in the art will appreciate that any platform may be used. For example, whole-exome sequencing data may be generated and analyzed to determine the genetic information.

The genetic information may be processed and/or “cleaned.” Taking NextSeq datasets as example, starting from 33,170 records data may be reviewed for includes. Because one may need large datasets to evaluate the low error rates, 11047 datasets with size <100 Mb (average 10 Mb) may be excluded. Because a method may rely on overlapping forward and reverse reads, 6,175 datasets with short reads (<70 bp for either forward or reverse reads) may be filtered. A similar strategy may be used for NovaSeq datasets as well.

The computing device 102 may receive the genetic information. For example, the computing device 102 may query the genetic information database 104 or the sequencing device 106. The computing device 102 may receive the genetic information via the network 108.

The computing device 102 may process the genetic information. For example, the computing device 102 may be configured to determine one or more error rates and/or error profiles. For example, example, the computing device 102 may be configured to determine an overall sequencer error rate; a top surface error rate and a bottom surface error rate at the flow-cell level; outlier tiles; one or more error rates associated with one or more reaction cycles. The computing device 102 may be configured to remove certain reaction cycles so as to suppress an error rate associated with one or more genomic loci. The computing device 102 may be configured to determine one or more error rates associated with one or more substitutions. For example, the computing device 102 may be configured to determine A>C, A>T, and C>G error types. The aforementioned substitutions and error types are merely exemplary and are not limiting. Any substitutions and any error types may be determined. The computing device 102 may be configured to determine that errors associated with a sequencer (e.g., the sequencer device 106) are associated with a pattern markedly distinct from PCR (polymerase chain reaction) errors.

For example, the computing device 102 may receive genetic information associated with paired-end sequencing. Paired-end sequencing may double sequencing yield by sequencing the input DNA molecule from both ends. However, when the input DNA molecule is short enough, such as equal to the sequencing length, the same DNA molecule will be sequenced twice (forward and reverse reads) in the sequencer. As such, identical readouts are expected if there are no sequencer errors. Therefore any discordance between forward and reverse reads must be from the sequencer. By using this method, error patterns associated with popular sequencing platforms, instruments, consumables (flowcells), and tiles in a flowcell were studied. The results provide critical insights to deep sequencing applications and future directions to improve instrument performances. Thus, by determining the number of errors that occur during paired end sequencing, the computing device may determine the errors attributable to the sequencing platform.

The network 108 may comprise any telecommunications network such as the Internet or a local area network. Other forms of communications can be used such as wired or wireless telecommunication channels, for example. The network 108 may be an optical fiber network, a coaxial cable network, a hybrid fiber-coaxial network, a wireless network, a satellite system, a direct broadcast system, an Ethernet network, a high-definition multimedia interface network, a Universal Serial Bus (USB) network, or any combination thereof. As a particular example, the network 108 can comprise a cellular network.

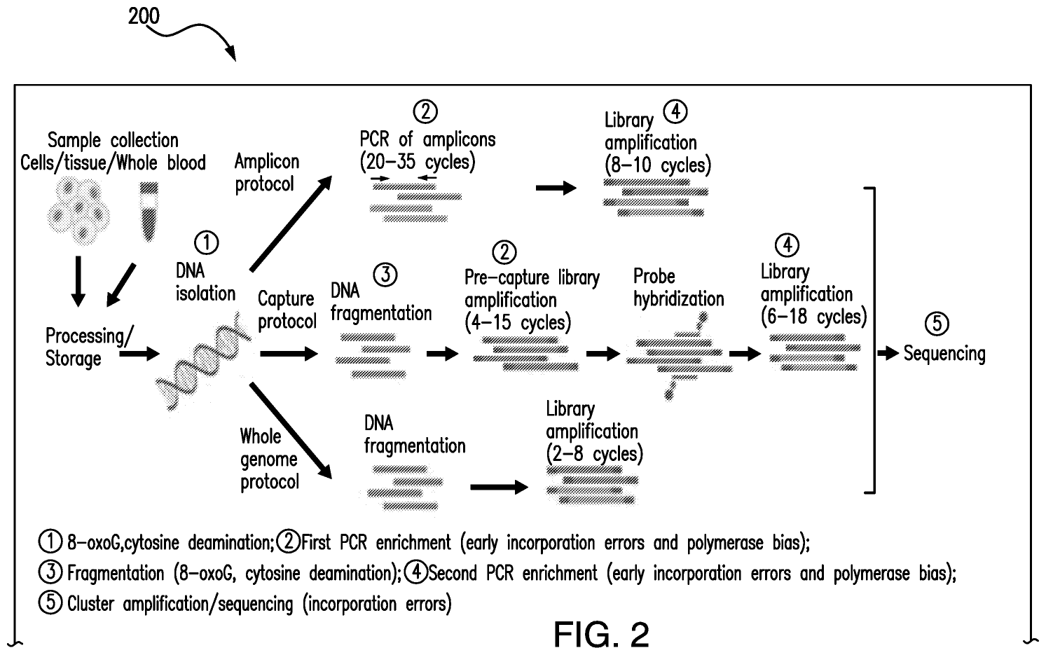

FIG. 2 shows an example typical NGS process flow 200. The workflow 200 may comprise multiple steps prior to sequencing, including sample processing, DNA isolation, and PCR amplification. Errors may be introduced in each of these steps. Spontaneous deamination of methylated cytosine to uracil can cause C>T/G>A errors. Additional errors can also be introduced by target-enrichment PCR and the sequencing step. Substitution error profiles may be determined by analyzing multiple sequencing datasets from one or more sequencing providers. To determine the lowest frequency at which a true somatic mutation can be distinguished from a sequencing error and to determine site-specific sequencing error rates, one may perform a dilution experiment using a matched cancer/normal cell line COLO829/COLO829BL (ATCC CRL-1974 and ATCC CRL-1980), both of which were established from the same patient: COLO829 was from malignant melanoma and COLO829BL was from the matching normal lymphoblastoid. Known somatic substitution mutations were targeted by amplicon sequencing (size of 130˜170 bp) on an Illumina HiSeq 2500 sequencer (abbreviated as HiSeq).

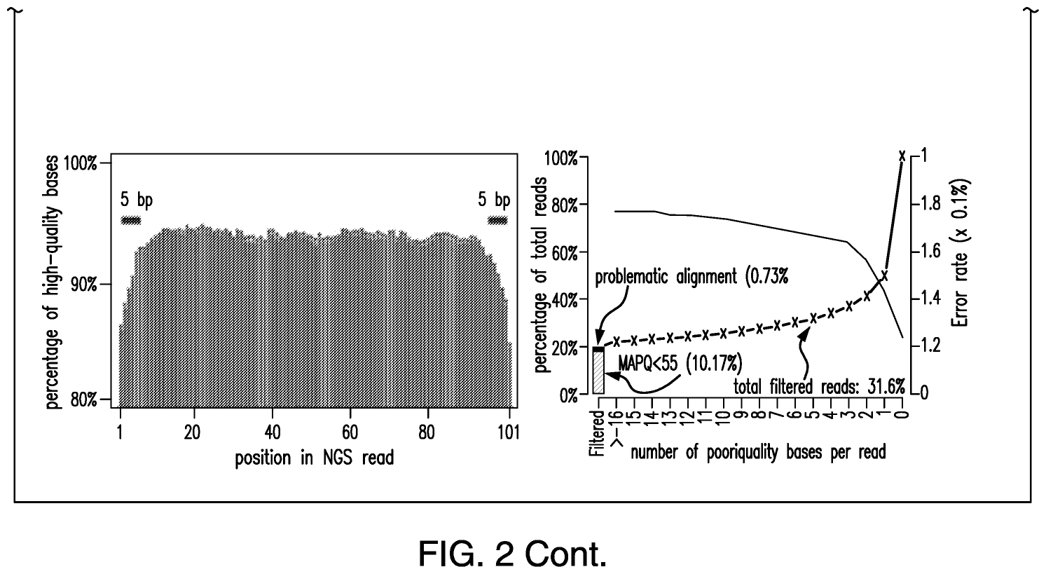

FIG. 3 shows an example process flow 300 for sequencing DNA. The method 300 may comprise analysis of sequencer errors by using reference DNA. Determining reference DNA may comprise starting from large number of cells (a), followed by limited PCR cycles and in turn sequencing. However, some cells may harbor bono fide mutations and inflate the sequencer error rate estimate. To minimize inter-cellular heterogeneity, one may start from minimal amount of starting DNA, followed by high PCR cycles and in turn sequencing (b). However, the high PCR cycles may introduce misincorporations and inflate the sequencer error rate. Here one may interrogate the sequencer errors by focusing on discordant based between forward and reverse reads of the same DNA segment within the overlapping regions. Such mismatches must have happened in sequencer. (d) Publicly available datasets associated with HiSeq, NextSeq, and NovaSeq in NCBI SRA, as of Oct. 20, 2019. Most datasets require permission to access resulted in limited datasets that are publicly available (e). Some datasets have lost read names during data submission, further reducing datasets for analysis.

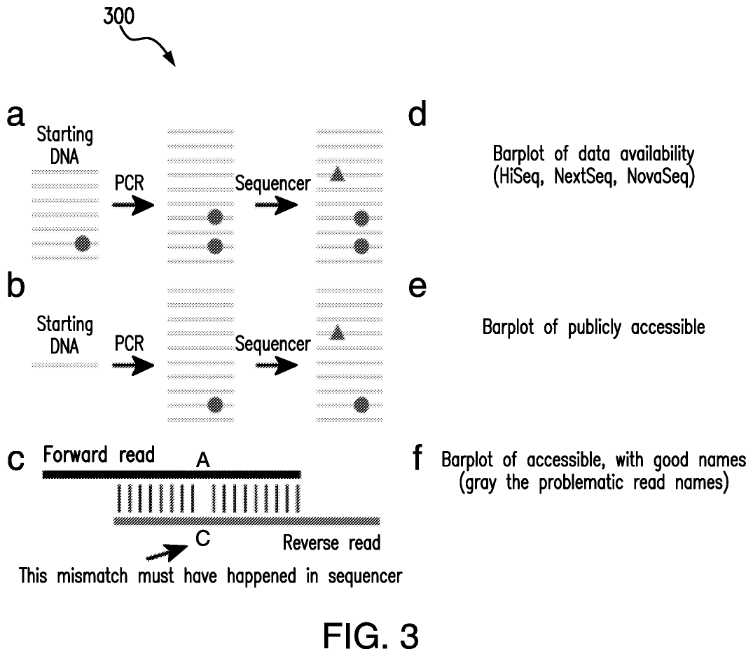

FIG. 4 shows an example analysis of sequencer errors (e.g., an error profile) 400 from a next generation sequencing process. The error profile 400 may comprise genetic information like base-pairs, error rates, and other relevant information. For example, FIG. 4 shows an analysis of sequencer errors in sections a-f. Tile-level error rate across representative flowcells for HiSeq (a,b), NextSeq (c,d), and NovaSeq (e,f) are shown as indicated. Shown in panels a, c and e are flowcells with “normal” error rate behavior, while shown corresponding flowcells with elevated overall error rate are shown in panels b, d, and f Each flowcell is partitioned into tiles according to surface, lane, swath (and camera for NextSeq). Error rates are in per million scale. Tile level error rates are capped at 200 for visualization purpose. In (g) the error rate (summarized as median across all tiles) distribution of common sequencing platforms is shown. Vertical bars indicate median (with value indicated) across all datasets. In (h) flowcell-level error rate distribution across instruments (with at least two flowcell experiments), are shown where instrument names are labeled for each platform, with the number of flowcells indicated in parentheses. Medians are indicated with a vertical black bar and on the left margin of the figure.

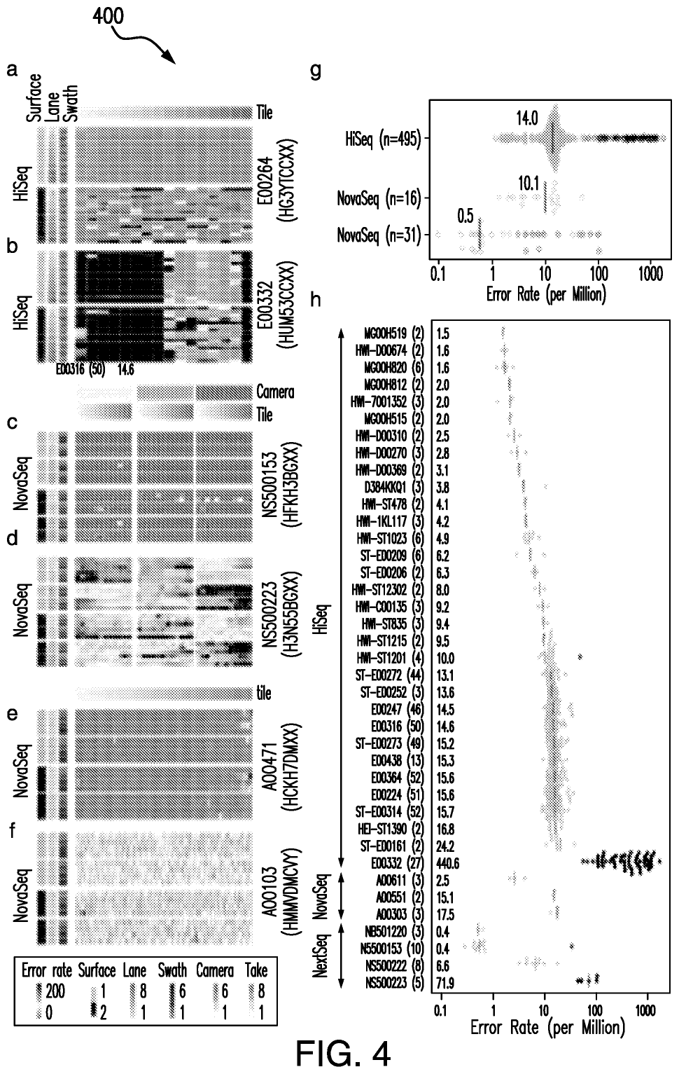

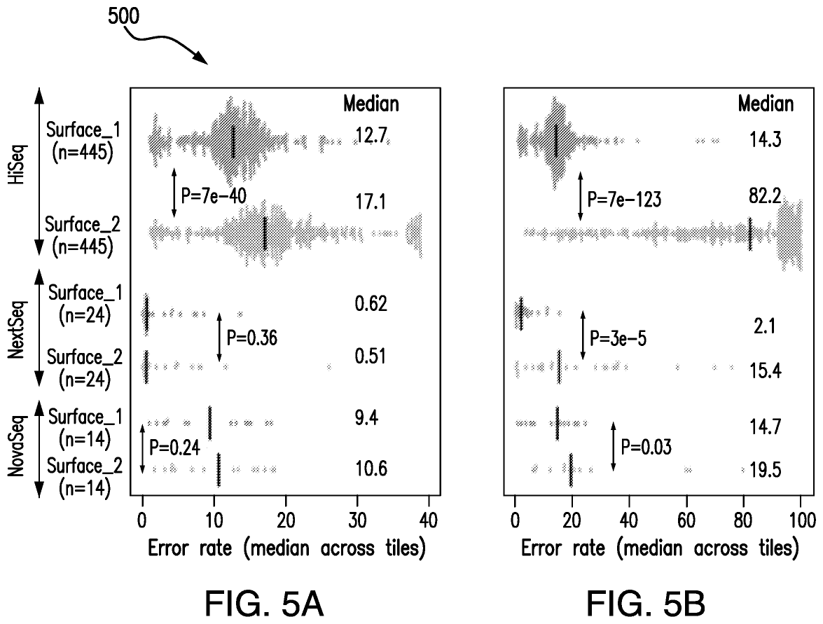

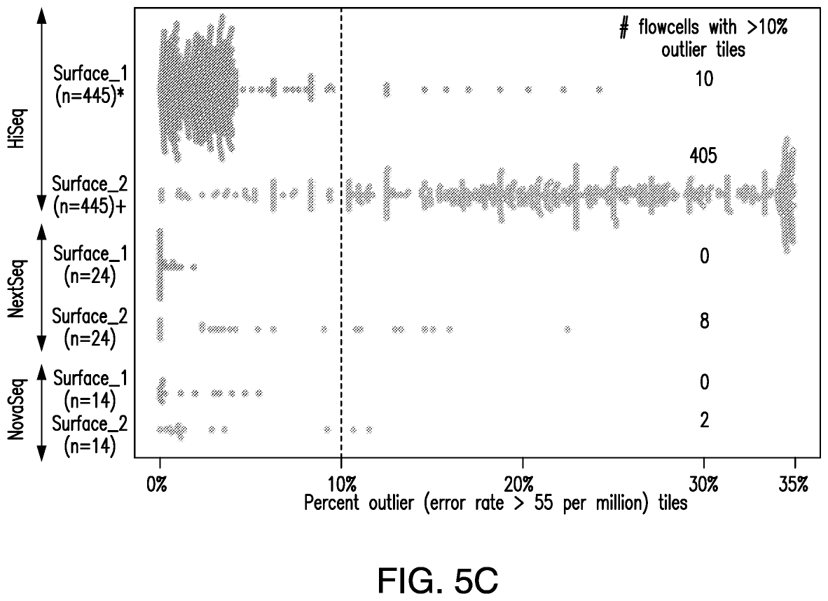

FIG. 5 shows an example flowcell analysis 500. For example, the flowcall analysis shows a comparison of error rate between top and bottom surfaces. Error rate of top and bottom surfaces are summarized by using median (a) and mean (b) across the flowcell. One-sided Wilcox rank sum P values are indicated for each platform. *: For HiSeq bottom surface, flowcell level error rate is capped at 40 (a) and 100 (b), respectively, for display purpose. (c) Prevalence of outlier tiles. Illustrated are percentages of outlier tiles with high error rate (>50 per million) for top (blue; Surface_1) and bottom (red; Surface_2) surfaces of each flowcell across three platforms, HiSeq, NextSeq, and NovaSeq. For each platform, number of flowcells with more than 10% high error rate tiles (dashed vertical line) are indicated by numbers on the right. *For HiSeq top surface, percentages <3% are replaced with a random number in [0%, 3%] for display purpose. +For HiSeq bottom surface, percentage >34% are replaced with a random number in [34%, 35%] for display purpose.

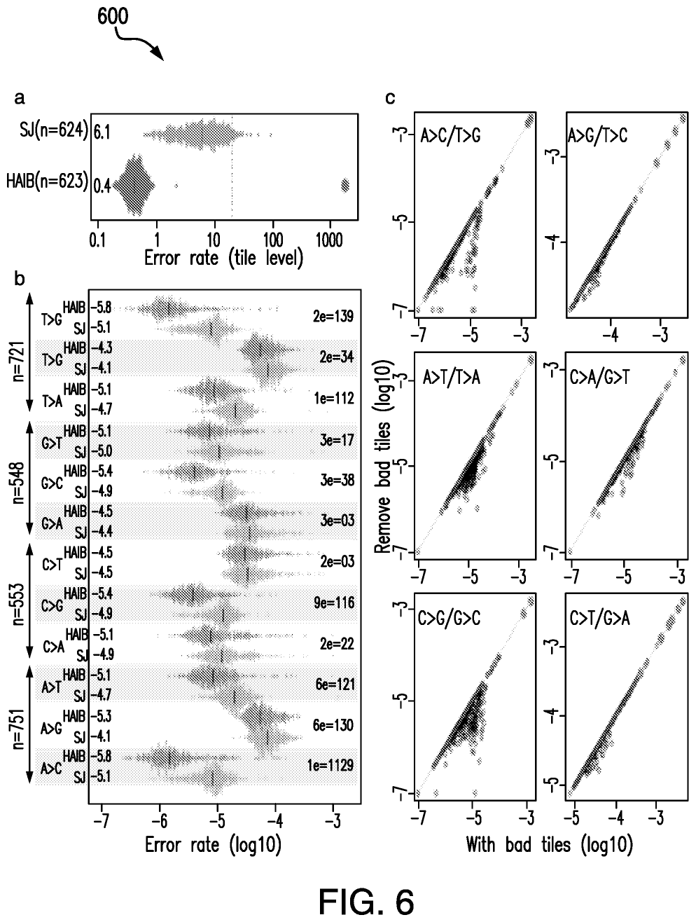

FIG. 6 shows an example overall sequencing error rate and sequencer error suppression scheme 600. In (a) two sequencers (HAIB: NovaSeq A00363 (blue) and SJ: NovaSeq A00214 (red)) have dramatically different tile-level error rate. Tiles with error rate >20 per million (vertical dashed line) are considered outlier tiles. In (b) the overall error rate from these two sequencers on the same DNA library is shown. Medians error rate (vertical black bars) of each misincorporation type are indicated in the left margin of the figure, and the one-sided Wilcox rank sum test P values between HAIB and SJ for each error types are indicated in the right margin of the figure. In (c) the effect of removing high-error rate tiles defined in (a) is shown. Each dot represents the site-specific error rate of given misincorporation types with (x-axis) and without (y-axis) the bad tiles. Red dots: spike-in true mutations. Diagonal (no change) is indicated with gray lines.

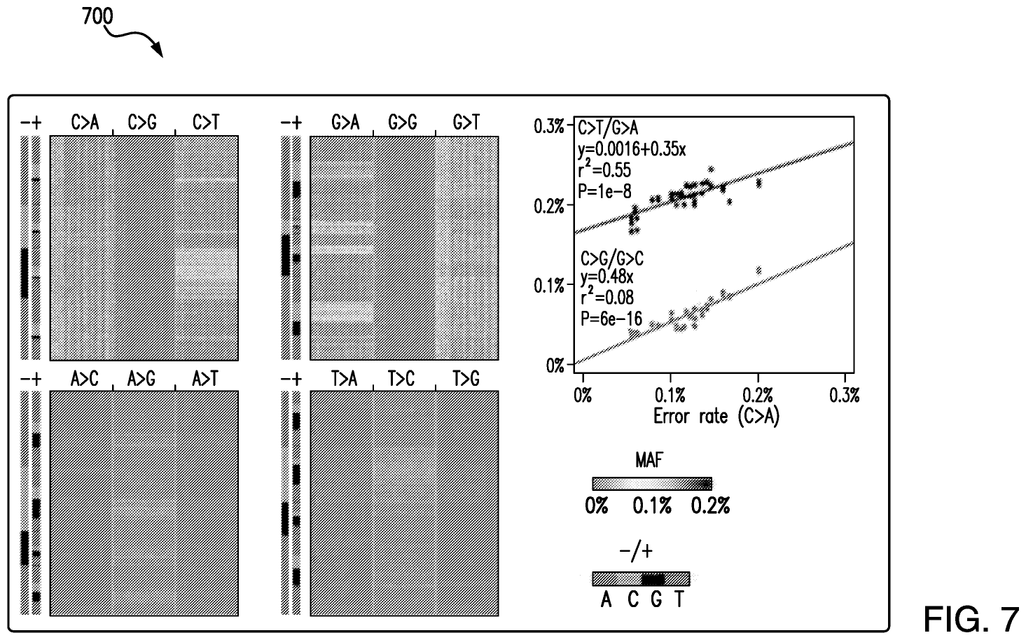

FIG. 7 shows an example sample level error profile 700. For example, to determine sample-level errors (as opposed to flowcell errors or tile level errors), which may indicate specimen handling/storage issues, a dataset of samples may be analyzed. For example, a heatmap may be generated so as to show the sequencing error rate in each sample (columns) stratified by sequence context associated with each substitution pattern (rows). As shown in FIG. 7, C>T/G>A errors exhibited a horizontal pattern across all samples, replicating the context dependency observed in the COLO829 dataset. By contrast, C>A/G>T errors exhibited a vertical (i.e., sample-specific) pattern regardless of sequence context, which may be attributable to sample-specific 8-oxoG stress. For example, C>A/G>T errors have been reported to be due to DNA damage during sample processing. For example, it also was found that C>A/G>T errors are enriched in a subset of samples, which indicates sub-optimal handling/storage conditions.

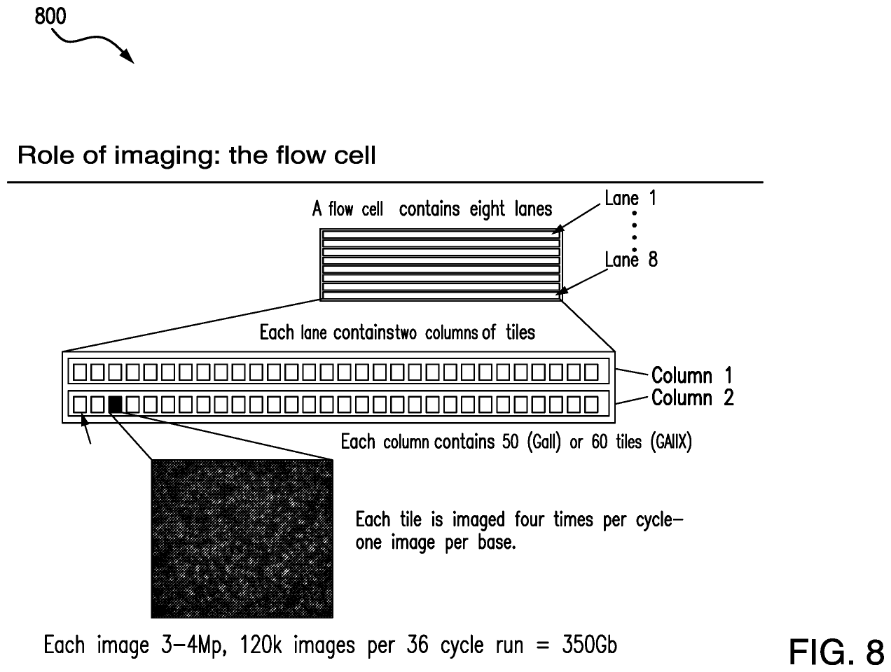

FIG. 8 shows an example imaging flow cell and flow cell image together 800. The imaging flow cell may comprise rows. The rows may comprise lanes. The lanes may comprise one or more columns, for instance, two columns. Each column may comprise one or more tiles. In an imaging process, each tile may be imaged one or more times. Each of the one or more tiles may comprise a top surface and a bottom surface as described herein. The top surface may be associated with a first error rate and the bottom surface may be associated with a second error rate.

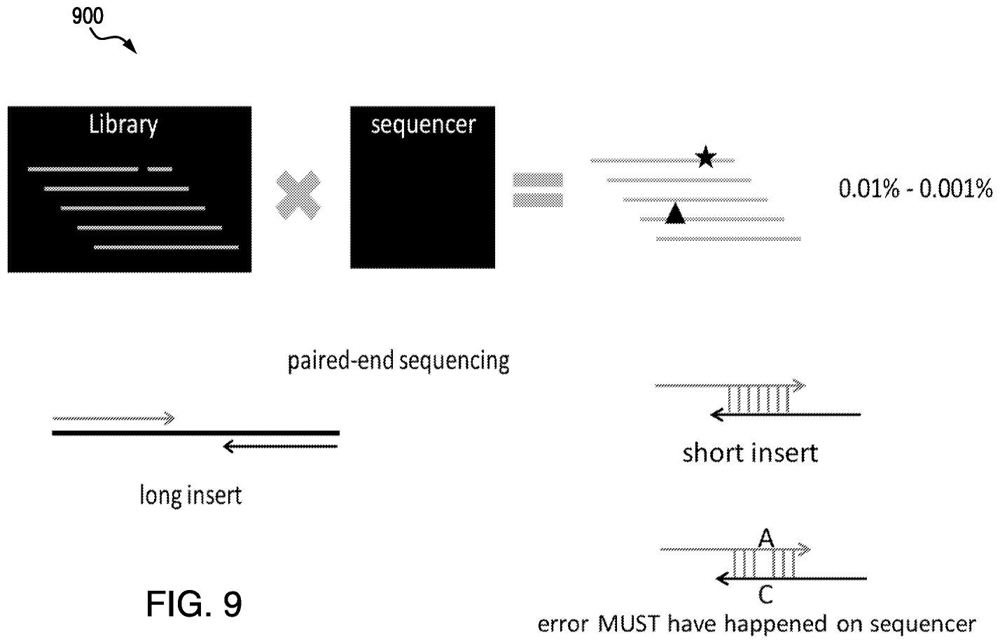

FIG. 9 shows an example sequencing process flow 900. The process flow 900 may involve components such as a library and a sequencer. Data from the library may be fed into the sequencer to generate an output. The process flow 900 may comprise paired-end sequencing. Paired-end sequencing may double sequencing yield by sequencing the input DNA molecule from both ends. However, when the input DNA molecule is short enough, such as equal to the sequencing length, the same DNA molecule will be sequenced twice (forward and reverse reads) in the sequencer. As such, identical readouts are expected if there are no sequencer errors. Therefore any discordance between forward and reverse reads must be from the sequencer. By using this method, error patterns associated with popular sequencing platforms, instruments, consumables (flowcells), and tiles in a flowcell were studied. The results provide critical insights to deep sequencing applications and future directions to improve instrument performances. Thus, by determining the number of errors that occur during paired end sequencing, the errors attributable to the sequencing platform may be determined.

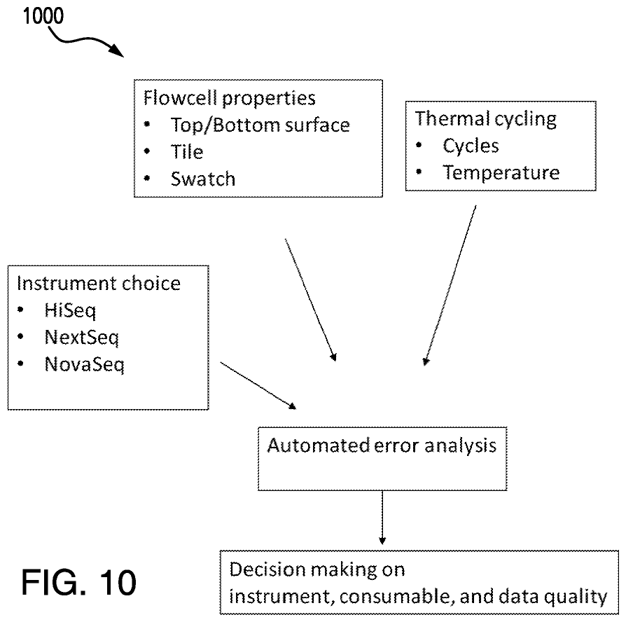

FIG. 10 shows an example process flow 1000. The process flow 1000 may comprise inputting flow cell properties, thermal cycling data such as cycles or temperature, instrument data such as instrument choice and other relevant information. The process flow 1000 may comprise automated error analysis. Further, the process flow 1000 may comprise decision making related to instruments, consumables, and data quality.

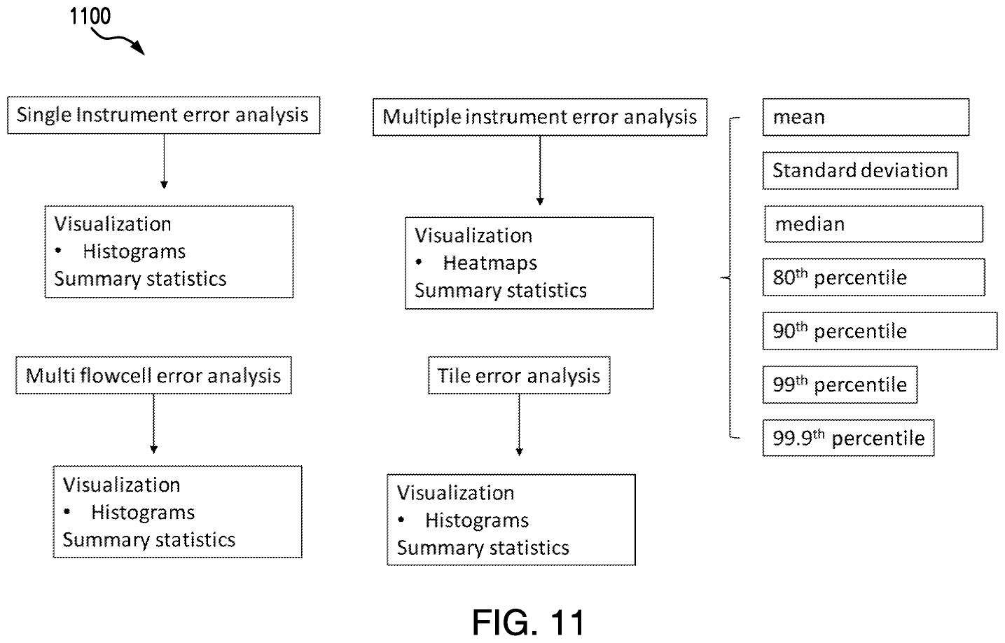

FIG. 11 shows an example process flow 1100. The process flow 1100 may comprise single instrument error analysis. The process flow 1100 may comprise visualization. The process flow 1100 may comprise multiflow error analysis. The process flow 1100 may comprise multiple instrument error analysis. The process flow 1100 may comprise tile error analysis. Visualization may comprise generating histograms, heatmaps, summary statistics and other relevant information such as means, medians, standard deviations and percentiles.

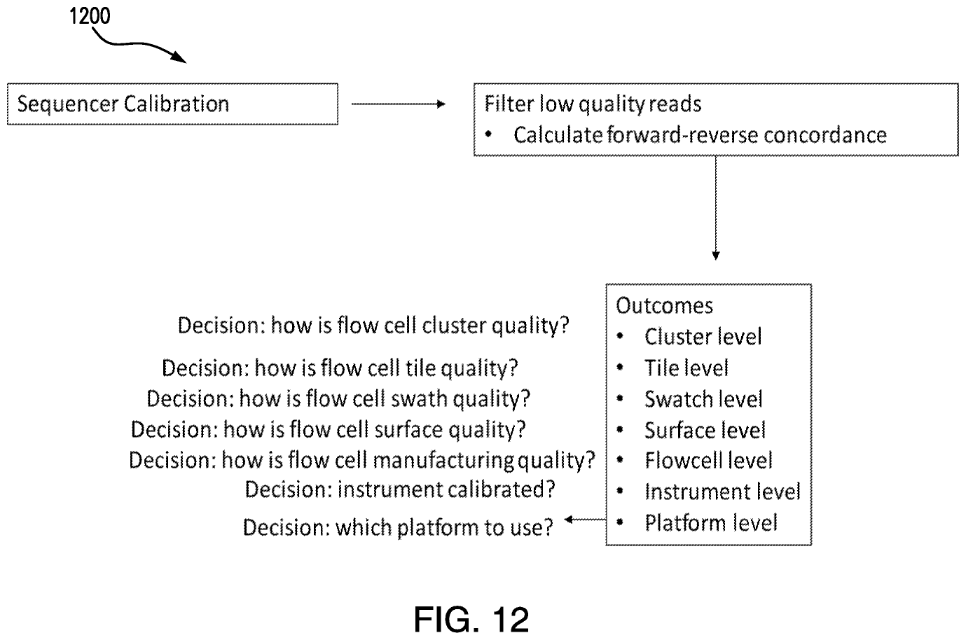

FIG. 12 shows an example process flow 1200. The process flow 1200 may comprise sequencer calibration. The process flow 1200 may comprise filtering low quality reads. Low quality reads may be filtered by calculating forward-reverse concordance. The process flow 1200 may comprise generating outcomes related to quality, quantity, fit, form, and other characteristics. Such outcomes may comprise data at the cluster level, tile level, swath level, surface level, flowcell level, instrument level, platform level and the like. For example, such data may comprise error rates associated with a cluster, a tile, a swath, a surface, a flowcell, and/or an instrument.

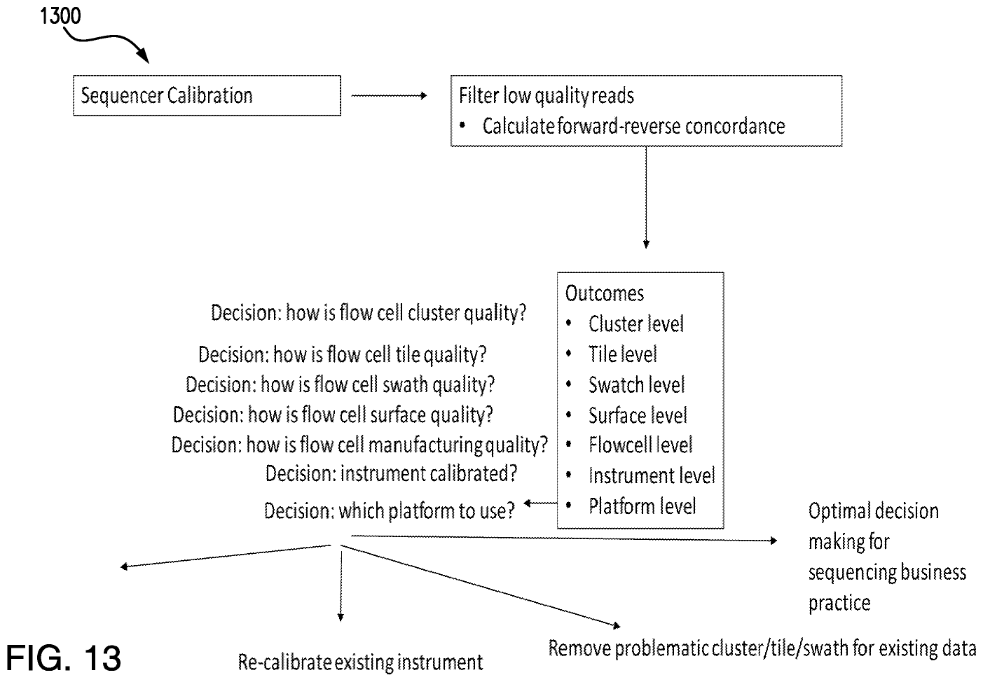

FIG. 13 shows an example process flow 1300. The process flow 1300 may comprise process flow 1200 with additional steps. For example, the process flow 1300 may comprise re-calibrating an existing instrument. The process flow 1300 may comprise optimal decision making for sequencing business practices. Further, the process flow 1300 may comprise removing problematic clusters, tiles, swathes, surfaces, flowcells, instruments, and/or platforms and the like. For example, an outliner cluster, and outlier tile, and outlier swathe, an outlier surface (e.g., top surface or bottom surface), an outlier flowcell, an outlier instrument, an outlier platform, combinations thereof, and the like may be determined. The modifier “outlier” may refer to a cluster, tile, swathe, surface, flowcell, instrument, platform, combinations thereof, and the like that are associated with error rate that falls outside the normal range.

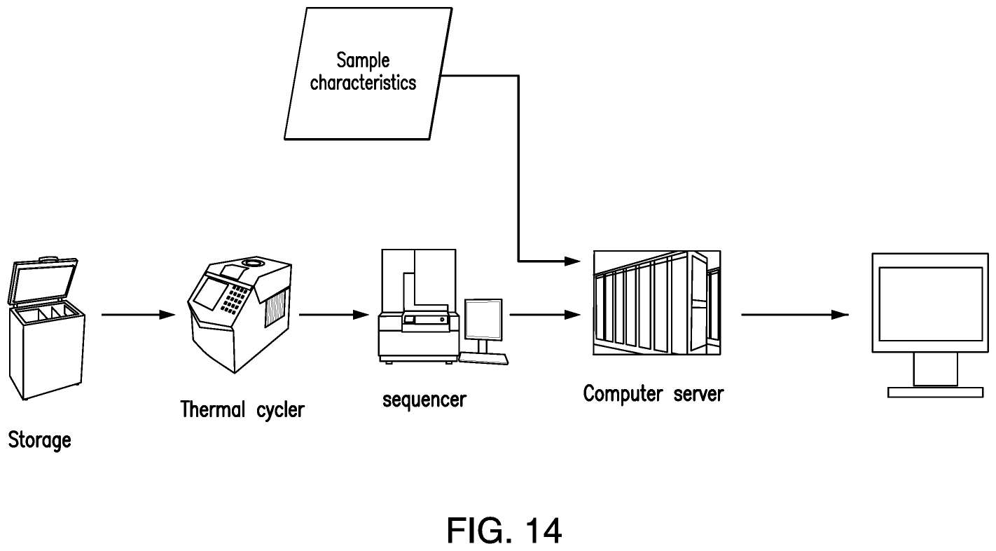

FIG. 14 shows an example process flow 1400. The process flow 1400 may comprise various components such as a storage, a thermal cycler, a sequencer, a computer server, a computer such as computer 1701, a display, and/or sample characteristics. The various components may be in communication with each other so as to facilitate the methods described herein. For example, the storage may store a sample. The storage may comprise, for example, a freezer. The sample may be transported to the a thermal cycler. The thermal cycler (also known as a thermocycler, PCR machine or DNA amplifier) may be configured to amplify segments of DNA via a polymerase chain reaction. The thermal cycler may be configured to facilitate other temperature-sensitive reactions, including restriction enzyme digestion, rapid diagnostics, other reactions, combinations thereof, and the like. The thermal cycler may comprise a thermal block. The thermal block may comprise holes where tubes holding the reaction mixtures can be inserted. The thermal cycler may be configured to raise and/or lower the temperature of the block in discrete, pre-programmed steps. The sample may then be sent to the sequencer. The sequencer may comprise, for example, an Illumina sequencer such as HiSeq, NovaSeq, and/or NextSeq. These examples are not limiting and any sequencer may be used. The sequencer may generate the genetic information. For example, the sequencer may generate a DNA sequence. The genetic information may be sent to, for example, the computer server and/or the computing device. The computing device may determine sample characteristics.

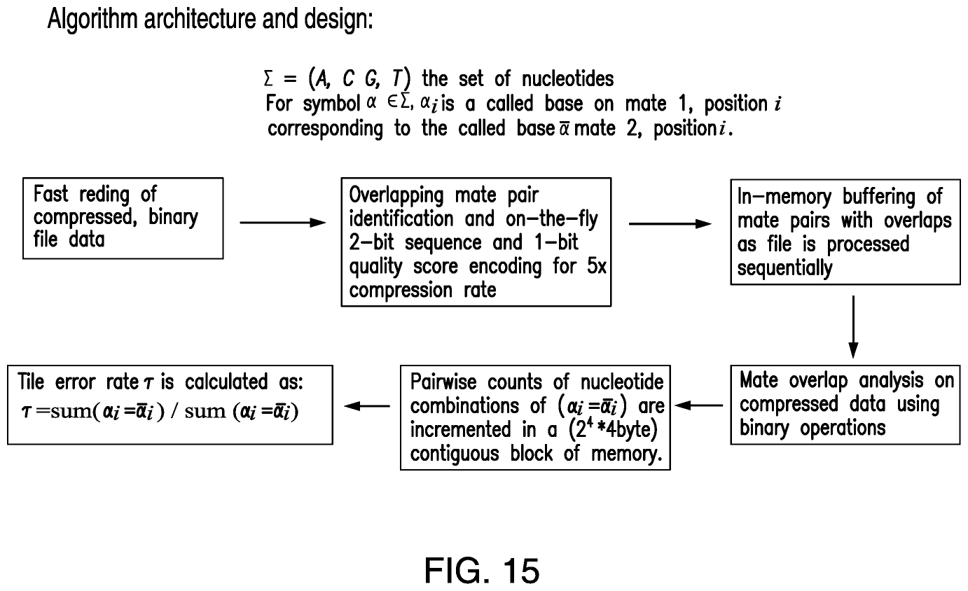

FIG. 15 shows an example process flow 1500. The process flow 1500 may comprise fast reading of a data file at 1510. For reference, “mate-pairs” may refer to a library construction methodology that allows both ends of a large fragment of DNA to be captured in the same template. This may comprise circularizing the large fragment, destroying the non-circular DNA, then re-fragmenting the circles. The junction may be biotinylated, allowing fragments containing those junctions to be captured. Adapters may be added to either end of this (smaller) fragment. Reads may be obtained from either end. For example, the data file may comprise the genetic information. For example, the data file may comprise a compressed, binary data file. The process flow 1500 may comprise identifying one or more overlapping mate pairs 1520. For example a first mate pair may be determined based on a forward read. For example, a second mate pair may be determined based on a reverse read. If the insert is shorter than twice the forward read length or the reverse read length, one or more overlapping mate pairs may be determined. The process flow 1500 may comprise performing on-the-fly two-bit sequencings at 1520. The process flow 1500 may comprise determining a one-bit quality score at 1520. The quality score may be associated with a sequence reading. For example, the quality score may be associated with a forward read. The quality score may be associated with a reverse read. The process flow 1500 may comprise storing the one or more mate pairs in storage (e.g., a buffer) at 1530. The mate pairs with overlaps may be buffered as the compressed, binary file is processed sequentially. The process flow 1500 may comprise performing a mate overlap analysis at 1540. For example, the mate overlap analysis may be performed on the compressed data using binary operations. The process flow 1500 may comprise determining pairwise counts of nucleotide combinations (a, ā) at 1550. The pairwise counts of nucleotide combination may be incremented in a 2⁴*4 byte contiguous memory block. The pairwise counts of nucleotide combinations may be used to determine a tile error rate at 1560. For example, the tile error rate τ may be determined as [symbol] τ=sum(a [symbol] _i≠a⁻_(i))/sum(a_i=a⁻_i) where Σ={A, C, G, T} is the set of nucleotides. For symbol a∈Σ, a_i is a called base on mate 1, position i corresponding to the called base a⁻ on mate 2, position i.

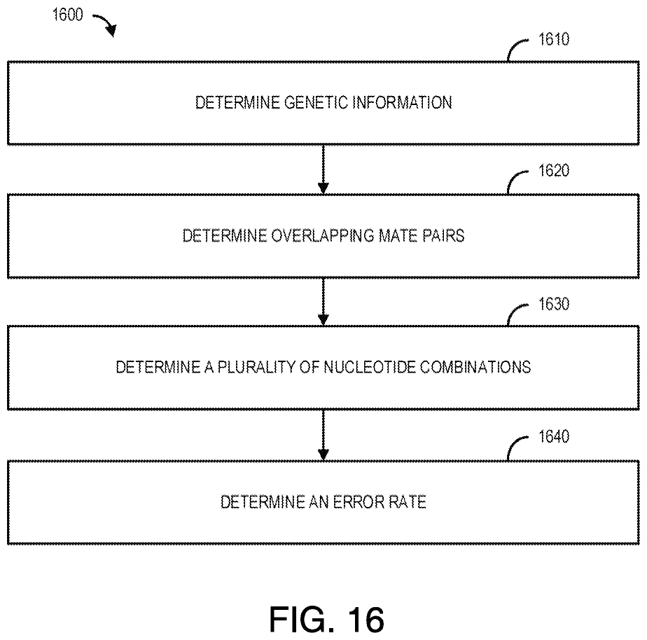

FIG. 16 shows an example method 1600. At step 1610, genetic information may be determined. The genetic information may comprise DNA, a DNA sequence, or the like. Determining genetic information may comprise receiving the genetic information from a database. For example, the genetic information may received from the genetic information database 104. Genetic information may comprise a library. The genetic information may comprise a dataset. For example, determining genetic information may comprise capturing a hybridization dataset. For example, for a hybridization-capture dataset, it may be required that there be >20,000 genomic sites for each of the 12 substitution types for a sample to be included in the analysis (21 of the 47 hybridization-capture samples passed this threshold). This requirement ensures that there are >20 genomic sites with error rate above the 99.9th percentile for each of the 12 substitution types. One advantage of using 99.9th percentile is that it automatically implies a false-positive rate of 0.1% (i.e., 99.9% of genomic sites have lower allele fraction than this statistic). Designed baits may be hybridized with adapter-ligated DNA libraries for 64 to 72 h. Then, the bait-target hybrids may be captured by streptavidin beads and enriched via secondary PCR enrichment.

For example, determining genetic information may comprise performing genetic sequencing. For example, genomic DNA may be sheared to ˜150- to 200-bp average size by using a Covaris LE220 focused ultrasonicator. The fragmented DNA may be end-repaired, dA-tailed, adapter-ligated, and enriched by PCR amplification using Kapa HTP library preparation kit Illumina 96rxn. Genetic sequencing may comprise performing paired-end cycles on a sequencing platform (e.g., the Illumina HiSeq X Ten system at 50,000×). This dataset has a median of 87,094 (range 31,437-129,934) base pairs covered at >15,000× across 47 samples. For example, whole-genome sequencing data may be analyzed by using CleanDeepSeq, as is known in the art, for each sample. To account for polymorphisms, within each sample, only loci with ≥20× coverage and >95% (so that binomial P value of observing 1 non-reference alleles from 20 reads is 4×10⁻⁵ and binomial P value of observing 2 non-reference alleles from 40 reads is 1.5×10⁻⁹ given the locus is heterozygous) reads being reference allele were merged into a single-count file. Loci with heterozygous calls (i.e., no alleles with fraction >95%) in any subject may be excluded from analysis. In an embodiment only loci with ≥20,000× collapsed coverage in the error analysis may be used.

The method 1600 may comprise comparing the effect of polymerases by using Q5 and Kapa polymerases to generate amplicon libraries. To ascertain enrichment PCR errors, this hybridization-capture dataset was also compared with an aggregated whole-genome sequencing dataset.

Determining genetic information may comprise processing genetic information. For example, determining the genetic information may comprise determining target regions. For example, a truth dataset may be composed of 19 somatic single-nucleotide variants (SNVs) from the matched cancer/normal cell lines COLO829 and COLO829BL, which may be derived from the same patient. To benchmark the variant detection limit, 0.1% and 0.02% of COLO829 (cancer) genomic DNA may be spiked into COLO829BL (normal) genomic DNA, resulting in two specimens diluted at 1:1000 and 1:5000, respectively, each with two replicates. The cancer and normal cell lines may be sequenced at 30,000× and 50,000×, respectively, to validate the wildtype status of sequences flanking the target SNVs in the cell lines. More importantly, the undiluted cancer cell line data may allow one to characterize false-positive detections from 1:1000 and 1:5000 dilution datasets because the mutant allele fraction of a false-positive call would not exhibit 1000- to 5000-fold increase in the undiluted cancer cell line. By plotting MAF in diluted versus undiluted samples of every position on the 18 amplicons, it may be determined that the only sites exhibiting this pattern of MAF increase were the 18 targeted variants. Therefore, it may be determined that no additional somatic variants exist in the 18 amplicons. The target SNVs may be selected by accounting for the genomic aneuploidy at chromosome 1q, which exhibits loss-of-heterozygosity (LOH) and has four copies in the cancer cell line. A selected somatic SNVs may include those with mutant alleles on 4 of 4, 2 of 4, or 1 of 4 copies of 1q, resulting in six distinct MAF levels (i.e., 0.01%, 0.02%, 0.04%, 0.05%, 0.1%, and 0.2%) over the two dilutions. HiSeq amplicon sequencing was carried out at respective depths of 300,000× and 1000,000× for the 1:1000 and 1:5000 dilution samples. It is noted that the allele fractions of the germline variants remain ˜0.5 in the dilution experiment because matched tumor/normal cell lines from the same individual may be used.

Determining genetic information may further comprise determining low quality reads. Low quality reads may comprise one or more sequencing reads with error rates that fall outside the normal range. For example in the HiSeq data, 92% of sequenced bases had a base quality score ≥30 that is, the estimated error rate was less than 0.1%. Reads were preprocessed (“Methods”) by trimming 5 bp at both ends of each read to remove potentially low-quality bases and possible adapter contamination. Reads with low-mapping quality may be removed. An association between the overall read quality and error rates of the remaining reads may be determined. The overall read quality may be measured as the total number of low-quality bases (defined as having a quality score ≤20, corresponding to an error rate of ≥1%) per read, and the error rate was measured by using the flanking bases in the amplicons as described above. Low quality reads (LQReads) may be defined as those with poor mapping quality or ≥5 low-quality bases. An in silico error suppression method was developed, CleanDeepSeq, to identify and filter the LQReads prior to allele counting. Since the target fragment size could be short (such as the 130 170 bp in the amplicon dataset), the forward and reverse reads in a paired-end sequencing setting may have significant overlaps. CleanDeepSeq was also designed to account for the concordance between forward and reverse readouts so that discordant readouts were not counted and concordant readouts were counted only once.

At step 1720, overlapping mate-pairs may be determined. For reference, “mate-pairs” may refer to a library construction methodology that allows both ends of a large fragment of DNA to be captured in the same template. This may comprise circularizing the large fragment, destroying the non-circular DNA, then re-fragmenting the circles. The junction may be biotinylated, allowing fragments containing those junctions to be captured. Adapters may be added to either end of this (smaller) fragment. Reads may be obtained from either end. For example, the overlapping mate-pairs may be determined according to the paired-end sequencing methodology. Paired-end sequencing may comprising sequencing the input DNA molecule from both ends. However, when the input DNA molecule is short enough, such as equal to the sequencing length, the same DNA molecule may be sequenced twice (forward and reverse reads) in the sequencer. Identical readouts are expected if there are no sequencer errors, therefore discordance between forward and reverse reads must be from the sequencer. For example, reads may be aligned by using bwa (0.7.12-r1039) with option “aln.” To avoid artifacts due to paralog mapping, one may include only base pairs in uniquely mappable regions for 100-mers (for hg19 and for hg38; downloaded March 2018) and for 75-mers. Only regions with a mappability score of 1 and length >300 bp were considered. Furthermore, the first and last 50 bp of a region may be excluded to account for potential edge effects. By using this method, error patterns associated with popular sequencing platforms, instruments, consumables (flowcells), and tiles in a flowcell may be determined.

At step 1730, a plurality of nucleotide combinations may be determined. The plurality of nucleotide combinations may comprise one or more nucleotides. For example, the one or more nucleotides may comprise adenine (A), thymine (T), guanine (G), and/or cytosine (C). The plurality of nucleotide combinations may comprise any combination of the one or more nucleotides. For example, a combination of the plurality of nucleotide combinations may comprise a TTT combination, a CTT combination, an ATT combination, an AAT combination, or any other possible combination. As one of skill will appreciate, the aforementioned combinations are merely exemplary and explanatory and are not restrictive. Determining the plurality of nucleotide combinations may comprise detecting one or more variants. For example, variant detection can be formulated into three related but distinct study designs. First, one may have a case-control design, where the sample of interest is compared against a control sample. Indeed, a simple combination with the existing deepSNV algorithm (which assumes case-control design) resulted in significant reduction (3- to 6-fold) of false positives by Clean-DeepSeq as compared to the standard pileup algorithm, without compromising sensitivity.

At step 1740, an error rate may be determined. The error rate may be associated with a device, a sample, a flow cell, a column, a tile, and/or PCR processing. For example, the error rate may be associated with an incorrect pair or substitution of the one or more nucleotides and/or plurality of nucleotide combinations. For example, an error may comprise an incorrect substitution. For example, an error may be associated with one or more substitution types. For example, the one or more substitution types may comprise A>C/T>G, C>A/G>T, C>G/G>C, A>G/T>C changes, A>C, A>T, and C>G error types. For example, determining the error rate may comprise determining an error rate and or substitution type associated with the one or more tiles. For example determining the error rate may comprise determining the error rate associated with the one or more flowcells. For example, for a given read pair, denote the total number of overlapping bases (between forward and reverse reads) as nᵣ, and the total number of sequenced bases in this overlapping region as 2nᵣ, where r=1, . . . , K and K represents the total number of reads in a given evaluation unit (such as tile, swath, lane, surface). Similarly, denote the total number of bases with a mismatch between forward and reverse reads as mᵣ. The total number of sequencer errors in this read pair in the overlapping region can be estimated as mᵣ. Note that the factor of 2 is not present as calculating the total number of sequenced bases, because at the low error frequency (e.g., <10⁻⁴), only one of the two different readouts is expected to be error. The tile-level error rate can be calculated as

$${e}_{t} = \frac{{\sum }_{r} {m}_{r}}{{\sum }_{r} 2 {n}_{r}}$$

where r∈Tile t
The flowcell level error rate is then defined as

e_{f}=average(eₜ|Tile t∈flowcell f)

where the average method is median unless otherwise stated. Determining the error rate may comprise comparing the error rate associated with flowcell surfaces (e.g., the top surface and/or the bottom surface) and outlier tiles. For example, the top surface may be associated with a lower error rate than bottom surface for some platforms. Alternatively, the top surface may be associated with a higher error rate than the bottom surface for some platforms For example, The top surfaces have significantly (P=) lower error rate than bottom surfaces for HiSeq, while for NovaSeq and NextSeq top surfaces tend to have lower error rate than bottom surfaces, though statistical significance is not reached. For this reason, the median error rate across all tiles in top and bottom surfaces, respectively, for each flowcell may be determined. Surface-level error rate may be re-calculated by taking mean error rate across all tiles in top and bottom surfaces, respectively. Outlier tiles may be determined according to the above. The outlier tiles at flowcell level for each platform, with stratification of top and bottom surfaces may be determined. For example, a tile is defined as an outlier tile if its error rate is >50 per million, with the observation that the essentially all flowcells have error rate <30 per million. Ten out of 445 (2%) HiSeq flowcells have more than 10% tiles with error rate >50 per million in the top surface, while in bottom surface 405 of 445 (91%) HiSeq flow cells have more than 10% tiles with error rate >50 per million. Interestingly, for 24 NextSeq flowcells the highest percentage is small in top surface, while 8 out of 24 (67%) NextSeq flowcells have more than 10% tiles with error rate >50 per million, indicating an improvement over HiSeq. For 14 NovaSeq flowcells, the highest percentage is only small in top surface, while only two out of 14 (14%) NovaSeq flowcells have more than 10% tile with error rate >50% per million. This data indicates a consistent improvement of bottom surface quality from HiSeq (91%) to NextSeq (67%) and NovaSeq (14%). The aforementioned methodology may be applied to larger analysis unit such as swath, or smaller analysis unit such as sub-regions in a given tile.

Determining the error rate may comprise determining a substitution error rate. For example, the substitution error rate may comprise an error rate associated with a mismatching of a nucleotide pair. For example, to determine the substitution error rate, one may take advantage of the high-depth sequencing data generated from the flanking sequences in the amplicons known to be devoid of genetic variations. Specifically, the substitution error rate for a given genomic site i was measured as follows:

$$error rate > mb ( \frac{1}{4} ) = \frac{\# reads with nucleotide at position i}{total \# reads at position i}$$

where g indicates the reference allele at genomic locus i and m represents each of the three possible substitutions caused by sequencing error. For example, at a given site with reference allele A, one may calculate error rates for the three possible mismatches A>C, A>G, and A>T, respectively. Please note that although the nomenclature “error rate” implies that the measured subject is caused by noise and the nomenclature “mutant allele fraction” (MAF) implies that the measured subject is a true somatic mutation, one may use both nomenclatures interchangeably because they may have the same formula.

One or more error profiles may be determined by performing a paired cancer-normal dilution experiment followed by deep sequencing. It may be determined that the substitution error rate can be suppressed computationally to 10⁻⁵ to 10⁻⁴. For example, the substitution error rate may be associated with one or more cancer-related substitutes and/or one or more hotspot substitutions. For example, the one or more cancer substitutions may be associated with somatic SNVs listed in COSMIC (v82; mostly adult cancers) are C>T/G>A mutations in high error rate contexts. To account for potential germline variants present in the COSMIC database, variants with a population allele fraction >0.1% (defined by the ExAC database) may be removed. It may be determined that 28.3% of COSMIC variants are in high error rate contexts. For pediatric cancers, 20.8% of somatic mutations (8% for neuroblastoma) are C>T/G>A mutations in high error rate contexts. Accordingly, it may be determined that >70% of the somatic substitutions are in low error rate contexts and that high-depth sequencing analysis can detect them at low (0.01 0.1%) frequency. Similarly, by using the list of hotspot substitutions, it may be determined that that 73% of hotspot substitutions are in low error rate contexts and high-depth sequencing analysis can detect them at low (0.01 0.1%) frequency.

Determining an error rate may comprise determining one or more errors introduced by specimen handling and/or specimen storage. These error rates may be described as “sample-level” errors because the errors present are generally found throughout the sample.

Determining the error rate may comprise determining a site-specific error rate. For example, The site-specific error rate may be calculated. The overall sequencing error is indeed lower than that of for A>C and T>G error types. On the other hand, the error rate is roughly comparable for other error types. This result indicates two possibilities: the sequencer might have enriched mis-incorporation types, such as A>C and T>G, or the reduction of sequencer errors has a negligable effect because PCR errors have rate ˜10-folder higher than that of sequencer errors, such as A>G and T>C errors. It may be determined that the C>A error rate is significantly correlated with that of C>G/G>C (linear regression P value=6×10⁻¹⁶) and C>T/G>A (linear regression P value=10⁻⁸), indicating that sample-specific DNA damage also contributes to an elevated error rate of C>G/G>C and C>T/G>A changes.

For example, the one or more error profiles may be attributed to different steps of NGS workflows, including sample handling, polymerase errors, and PCR enrichment steps as described herein. For example, determining the error rate may comprise determining one or more errors associated with one or more instruments or devices. For example, determining the one or more errors associated with one or more instruments or devices may comprise determining one or more error profiles. For example, because it appears that different flowcells tend to have consistent error rates within the same instrument, the effect of different instruments on the overall sequencing error rate, without determining flowcells effect. For example, where exactly the same DNA libraries were sequenced on two different NovaSeq instruments (such as on the COLO829 dilution datasets published previously, these two instruments may have an error rate difference, by which one may expect the data generated to have lower error rate.

Determining the error rate may comprise determining errors associated with polymerase chain-reaction enrichment. For example, the errors introduced by enrichment PCR (6-18 cycles) may be studied. For example, the sequencing data of 1663 whole genomes that had undergone first-enrichment PCR may be aggregated. The hybridization-capture sequencing dataset, which underwent two enrichment PCR rounds, may be compared to WGS dataset with CleanDeepSeq. Both datasets may be sequenced by using Illumina X Ten. One may find a statistically significant linear relationship between hybridization-capture targeted sequencing data and WGS data among the 12 error types, and a ˜5.5- to 6.5-fold increase in errors may be observed in capture sequencing data. For example, the method may comprise comparing the effect of polymerases by using Q5 and Kapa polymerases to generate amplicon libraries (“Methods”), which were sequenced on the latest Illumina sequencing platform NovaSeq 6000 (abbreviated as NovaSeq) at both St. Jude Children's Research Hospital and HudsonAlpha Institute of Biotechnology sequencing centers. To study the effect of sample-level damages, a high-depth sequencing (˜50,000× coverage) dataset generated by hybridization-capture of 47 leukemia samples was used. To ascertain enrichment PCR errors, this hybridization-capture dataset was also compared with an aggregated whole-genome sequencing dataset.

The method 1700 may further comprise suppressing errors. For example, because the base quality dropped at read ends for HiSeq data, the first and last five base pairs of the reads may be trimmed. This trimming may also clean up potential residual adapter/primer sequences. The same parameter may be used for other datasets as well. To avoid artifacts attributable to mapping ambiguity, a stringent mapping quality cut off may be used (MAPQ) cutoff of 55 (value 255 also discarded because it indicates that the mapping quality is not available which affected 18.2% of reads (16.2% if using a MAPQ cutoff of 30 in the HiSeq dataset. Furthermore, because reads with insertion/deletions and/or structural rearrangements may introduce alignment ambiguity, only reads with substitution mismatches may be included (i.e., the CIGAR string matches the regular expression/\hat{ }\d+M$/; affecting ˜1% reads. Reads with ≥5% bases of Phred quality score <20 may also be suppressed because they have elevated error rates. To avoid counting an allele from the same DNA fragment twice, the following procedure for fragments with overlapping read pairs may be used: (i) if a base pair has only one readout in either forward or reverse read (non-overlapping part), it may only be counted as 1 if its Phred quality score is ≥30; (ii) if a base pair has two readouts in both forward and reverse reads (overlapping part), it may be counted as 1 if forward and reverse readouts are concordant and both have Phred quality score ≥30 or if only one readout has Phred quality score ≥30. The effect of removing tiles with high error rates may be determined. Site-specific error rate may be determined. For example, A>C and G>T error sites have error rate reduction of between 2-fold and 10-fold, followed by C>G/G>C errors with ˜3-fold reduction. Using standard pileup, the error rate of both Exo-me WGA and Exome Native is ˜0.1% (log 10 scale of −3 in the left panel of FIG. 6a, b), consistent with previous reports. Applying CleanDeepSeq, as is known in the art, resulted in a 10-fold reduction of error rate (˜0.01%, log 10 scale of 4) in both datasets.

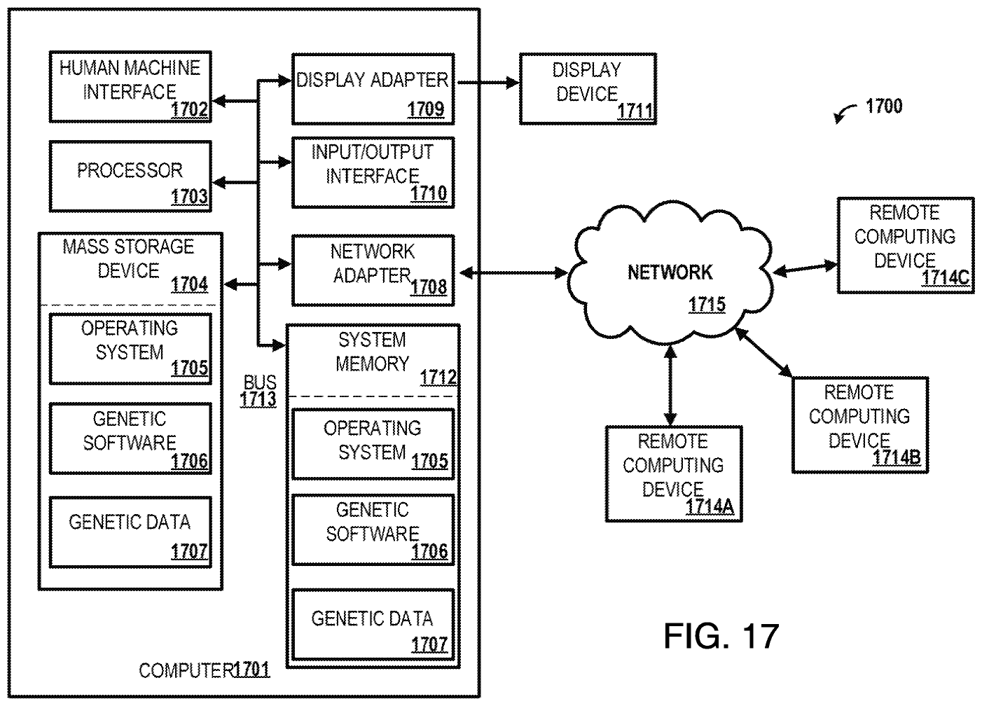

FIG. 17 shows a system 1700 for intermediate data object generation and use. A computer 1701 may comprise one or more processors 1703, a system memory 1712, and a bus 1713 that couples various components of the computer 1701 including the one or more processors 1703 to the system memory 1712. In the case of multiple processors 1703, the computer 1701 may utilize parallel computing.

The bus 1713 may comprise one or more of several possible types of bus structures, such as a memory bus, memory controller, a peripheral bus, an accelerated graphics port, and a processor or local bus using any of a variety of bus architectures.

The computer 1701 may operate on and/or comprise a variety of computer readable media (e.g., non-transitory). Computer readable media may be any available media that is accessible by the computer 1701 and comprises, non-transitory, volatile and/or non-volatile media, removable and non-removable media. The system memory 1712 has computer readable media in the form of volatile memory, such as random access memory (RAM), and/or non-volatile memory, such as read only memory (ROM). The system memory 1712 may store data such as genetic data 1707 and/or program modules such as operating system 1705 and genetic software 1706 that are accessible to and/or are operated on by the one or more processors 1703.

The computer 1701 may also comprise other removable/non-removable, volatile/non-volatile computer storage media. The mass storage device 1704 may provide non-volatile storage of computer code, computer readable instructions, data structures, program modules, and other data for the computer 1701. The mass storage device 1704 may be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Any number of program modules may be stored on the mass storage device 1704. An operating system 1705 and genetic software 1706 may be stored on the mass storage device 1704. One or more of the operating system 1705 and genetic software 1706 (or some combination thereof) may comprise program modules and the genetic software 1706. Genetic data 1707 may also be stored on the mass storage device 1704. Data 1707 may be stored in any of one or more databases known in the art. The databases may be centralized or distributed across multiple locations within the network 1715.

A user may enter commands and information into the computer 1701 via an input device (not shown). Such input devices comprise, but are not limited to, a keyboard, pointing device (e.g., a computer mouse, remote control), a microphone, a joystick, a scanner, tactile input devices such as gloves, and other body coverings, motion sensor, and the like These and other input devices may be connected to the one or more processors 1703 via a human machine interface 1702 that is coupled to the bus 1713, but may be connected by other interface and bus structures, such as a parallel port, game port, an IEEE 1394 Port (also known as a Firewire port), a serial port, network adapter 1708, and/or a universal serial bus (USB).

A display device 1711 may also be connected to the bus 1713 via an interface, such as a display adapter 1709. It is contemplated that the computer 1701 may have more than one display adapter 1709 and the computer 1701 may have more than one display device 1711. A display device 1711 may be a monitor, an LCD (Liquid Crystal Display), light emitting diode (LED) display, television, smart lens, smart glass, and/or a projector. In addition to the display device 1711, other output peripheral devices may comprise components such as speakers (not shown) and a printer (not shown) which may be connected to the computer 1701 via Input/Output Interface 1710. Any step and/or result of the methods may be output (or caused to be output) in any form to an output device. Such output may be any form of visual representation, including, but not limited to, textual, graphical, animation, audio, tactile, and the like. The display 1711 and computer 1701 may be part of one device, or separate devices.

The computer 1701 may operate in a networked environment using logical connections to one or more remote computing devices 1714A,B,C. A remote computing device 1714A,B,C may be a personal computer, computing station (e.g., workstation), portable computer (e.g., laptop, mobile phone, tablet device), smart device (e.g., smartphone, smart watch, activity tracker, smart apparel, smart accessory), security and/or monitoring device, a server, a router, a network computer, a peer device, edge device or other common network node, and so on. A remote computing device 1714A,B,C may, for example, be a genetic information database or sequencing device. Logical connections between the computer 1701 and a remote computing device 1714A,B,C may be made via a network 1715, such as a local area network (LAN) and/or a general wide area network (WAN). Such network connections may be through a network adapter 1708. A network adapter 1708 may be implemented in both wired and wireless environments. Such networking environments are conventional and commonplace in dwellings, offices, enterprise-wide computer networks, intranets, and the Internet.

Application programs and other executable program components such as the operating system 1705 are shown herein as discrete blocks, although it is recognized that such programs and components may reside at various times in different storage components of the computing device 1701, and are executed by the one or more processors 1703 of the computer 1701. An implementation of software 1706 may be stored on or sent across some form of computer readable media. Any of the disclosed methods may be performed by processor-executable instructions embodied on computer readable media.

While specific configurations have been described, it is not intended that the scope be limited to the particular configurations set forth, as the configurations herein are intended in all respects to be possible configurations rather than restrictive.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that an order be inferred, in any respect. This holds for any possible non-express basis for interpretation, including: matters of logic with respect to arrangement of steps or operational flow; plain meaning derived from grammatical organization or punctuation; the number or type of configurations described in the specification.

It will be apparent to those skilled in the art that various modifications and variations may be made without departing from the scope or spirit. Other configurations will be apparent to those skilled in the art from consideration of the specification and practice described herein. It is intended that the specification and described configurations be considered as exemplary only, with a true scope and spirit being indicated by the following claims.

What is claimed is:

1. A method comprising:
determining genetic information;
determining, based on the genetic information, overlapping mate pairs, wherein the overlapping mate pairs are associated with a sequence and a quality score;
determining, based on the overlapping mate pairs, at least one of a plurality of nucleotide combinations, wherein the at least one of the plurality of nucleotide combinations is associated with the sequence and the quality score;
determining, based on the at least one of the plurality of nucleotide combinations, an error rate.

2. The method of claim 1, further comprising determining a source of an error.

3. The method of claim 2, wherein determining the source of the error comprises identifying a device associated with an error profile.

4. The method of claim 2, wherein determining the source of an error comprises determining at least one nucleotide combination associated with an error profile.

5. The method of claim 1, wherein the genetic information comprises at least one DNA sequence.

6. The method of claim 1, wherein the sequence comprises at least one base pair.

7. The method of claim 1, wherein the quality score comprises a read value.

8. A system comprising:
a sequencing device configured to:
determine genetic information;
transmit genetic information; and
a computing device configured to:
receive genetic information;
determine, based on the genetic information, overlapping mate pairs, wherein the overlapping mate pairs are associated with a sequence and a quality score;
determine, based on the overlapping mate pairs, at least one of a plurality of nucleotide combinations;
determining, based on the at least one of the plurality of nucleotide combinations, an error rate.

9. The system of claim 8, wherein the computing device is further configured to determine a source of an error.

10. The system of claim 9, wherein, to determine the source of error, the computing device is further configured to determine a device associated with an error profile.

11. The system of claim 8, wherein, to determine the source of error, the computing device is further configured to determine at least one nucleotide combination associated with an error profile.

12. The system of claim 8, wherein the genetic information comprises at least one DNA sequence.

13. The system of claim 8, wherein the sequence comprises at least one base pair.

14. The system of claim 8, wherein the quality score comprises a read value.

15. An apparatus comprising:
one or more processors; and
memory storing processor executable instructions that, when executed by the one or more processors, cause the apparatus to:
determining genetic information;
determining, based on the genetic information, overlapping mate pairs, wherein the overlapping mate pairs are associated with a sequence and a quality score;
determining, based on the overlapping mate pairs, at least one of a plurality of nucleotide combinations;
determining, based on the at least one of the plurality of nucleotide combinations, an error rate.

16. The apparatus of claim 15, wherein the processor executable instructions, when executed by the one or more processors, further cause the apparatus to determine a source of an error.

17. The apparatus of claim 15, wherein the processor executable instructions that, when executed by the one or more processors, cause the apparatus to determine the source of the error further cause the apparatus to identify a device associated with an error profile.

18. The apparatus of claim 15, wherein the processor executable instructions that, when executed by the one or more processors, cause the apparatus to determine the source of the error further cause the apparatus to identify a nucleotide combination associated with an error profile.

19. The apparatus of claim 15, wherein the genetic information comprises at least one DNA sequence.

20. The apparatus of claim 15, wherein the sequence comprises at least one base pair.
